(19) United States
(12) Patent Application Publication
(10) Pub. No.: US 2025/0359726 A1
(43) Pub. Date: Nov. 27, 2025

(54) MEDICAL APPARATUS, MEDICAL SYSTEM, CONTROL METHOD, AND COMPUTER-READABLE RECORDING MEDIUM

(71) Applicant: OLYMPUS MEDICAL SYSTEMS CORP., Tokyo (JP)

(72) Inventors: Yasuo TANIGAMI, Tokyo (JP); Yusuke OTSUKA, Yokohama-shi (JP); Noriko KURODA, Tokyo (JP); Takaaki IGARASHI, Tokyo (JP); Maho NISHIYAMA, Tokyo (JP); Kotone NAKAJIMA, Tokyo (JP)

(73) Assignee: OLYMPUS MEDICAL SYSTEMS CORP., Tokyo (JP)

(21) Appl. No.: 19/289,375

(22) Filed: Aug. 4, 2025

Related U.S. Application Data
(63) Continuation of application No. PCT/JP2023/004398, filed on Feb. 9, 2023.

(51) Int. Cl. A61B 1/00; A61B 1/04; A61B 1/06; G06T 7/00

(52) U.S. Cl. CPC A61B 1/00009; A61B 1/0002; A61B 1/0005; A61B 1/043; A61B 1/0638; A61B 1/0661; G06T 7/0014; G06T 2207/10064; G06T 2207/10068; G06T 2207/30092; G06T 2207/30096

(57) ABSTRACT

A medical apparatus includes: a processor including hardware, the processor being configured to acquire area information that includes information on an area of interest based on input from a user, the area of interest being an organ that is adjacent to a target organ that is a target for thermal treatment, acquire a fluorescence image that is generated based on an imaging signal captured at a timing of application of excitation light, extract a thermally denatured area based on the fluorescence image, and generate thermal denaturation information based on the area of interest and the thermally denatured area.

### CROSS REFERENCES TO RELATED APPLICATIONS

This application is a continuation of International Application No. PCT/JP2023/004398, filed on Feb. 9, 2023, the entire contents of which are incorporated herein by reference.

### BACKGROUND

### 1. Technical Field

The present disclosure relates to a medical apparatus, a medical system, a control method, and a computer-readable recording medium.

### 2. Related Art

In the related art, a technology for visualizing a thermally denatured state of a living tissue at the time of treatment on the living tissue by an energy device or the like is known (for example, see International Publication No. 2020/054723).

In the technology described in International Publication No. 2020/054723, a thermally denatured state of a living tissue is visualized based on a captured image in which fluorescence that is emitted from the living tissue due to application of excitation light to the living tissue is imaged. Specifically, in the technology described in International Publication No. 2020/054723, an area in which fluorescence intensity is higher than certain fluorescence intensity that is set in advance among all of pixels in the captured image is displayed as an area in which thermal denaturation is high.

### SUMMARY

In some embodiments, a medical apparatus includes: a processor including hardware, the processor being configured to acquire area information that includes information on an area of interest based on input from a user, the area of interest being an organ that is adjacent to a target organ that is a target for thermal treatment, acquire a fluorescence image that is generated based on an imaging signal captured at a timing of application of excitation light, extract a thermally denatured area based on the fluorescence image, and generate thermal denaturation information based on the area of interest and the thermally denatured area.

In some embodiments, a medical system includes: an endoscope that includes an image sensor; a light source apparatus that includes a light source configured to apply white light and excitation light; and a control apparatus that includes a processor comprising hardware, the processor being configured to acquire area information that includes information on an area of interest based on input from a user, the area of interest being an organ that is adjacent to a target organ that is a target for thermal treatment, acquire a fluorescence image that is generated based on an imaging signal captured at a timing of application of excitation light, extracts a thermally denatured area based on the fluorescence image, and generate thermal denaturation information based on the area of interest and the thermally denatured area.

In some embodiments, provided is a control method of controlling a medical apparatus. The control method includes: acquiring area information that includes information on an area of interest based on input from a user, the area of interest being an organ that is adjacent to a target organ that is a target for thermal treatment; acquiring a fluorescence image that is generated based on an imaging signal captured at a timing of application of excitation light; extracting a thermally denatured area based on the fluorescence image; and generating thermal denaturation information based on the area of interest and the thermally denatured area.

In some embodiments, provided is a non-transitory computer-readable recording medium with an executable program stored thereon. The program causes a processor of a medical device to execute: acquiring area information that includes information on an area of interest based on input from a user, the area of interest being an organ that is adjacent to a target organ that is a target for thermal treatment; acquiring a fluorescence image that is generated based on an imaging signal that is captured at a timing of application of excitation light; extracting a thermally denatured area based on the fluorescence image; and generating thermal denaturation information based on the area of interest and the thermally denatured area.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating an overall configuration of an endoscope system according to one embodiment;

FIG. 2 is a block diagram illustrating a functional configuration of a main part of the endoscope system according to one embodiment;

FIG. 3 is a diagram illustrating a wavelength characteristic of excitation light that is emitted by a second light source unit;

FIG. 4 is a diagram illustrating transmission characteristics of a cut filter;

FIG. 5 is a diagram for explaining an observation principle in a fluorescence observation mode;

FIG. 6 is a diagram for explaining an observation principle in a normal light observation mode;

FIG. 7 is a flowchart indicating a control method that is implemented by a control apparatus;

FIG. 8 is a diagram for explaining the control method;

FIG. 9 is a diagram for explaining the control method;

FIG. 10 is a diagram for explaining the control method;

FIG. 11 is a diagram for explaining a modification of one embodiment; and

FIG. 12 is a diagram for explaining a modification of one embodiment.

### DETAILED DESCRIPTION

Modes (hereinafter, “embodiments”) for carrying out the disclosure will be described below with reference to the drawings. Meanwhile, the disclosure is not limited by the embodiments described below. Further, in description of the drawings, the same components are denoted by the same reference symbols.

### Overall Configuration of Endoscope System

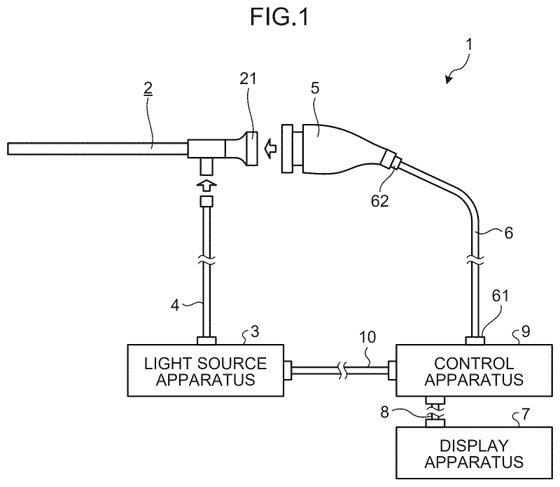

FIG. 1 is a diagram illustrating an overall configuration of an endoscope system 1 according to one embodiment.

The endoscope system 1 according to one embodiment is an endoscope system that is used for surgical operation for stomach cancer or the like. Specifically, in the endoscope system 1, an insertion portion 2 is inserted into a body of a subject, an image of an observation area including a region in which thermal treatment is performed by an energy device or the like inside the subject is captured, and a display image that is based on captured image data is displayed on a display apparatus 7. An operator performs the thermal treatment by the energy device or the like while checking the display image.

As illustrated in FIG. 1, the endoscope system 1 includes the insertion portion 2, a light source apparatus 3, a light guide 4, a camera head 5, a first transmission cable 6, the display apparatus 7, a second transmission cable 8, a control apparatus 9, and a third transmission cable 10.

The insertion portion 2 is rigid or partly flexible, has a thin and elongated shape, and is inserted into the subject (into a urinary bladder). Further, an optical system, such as a lens, for forming an object image is arranged inside the insertion portion 2.

The light source apparatus 3 is connected to one end of the light guide 4, and supplies illumination light, which is to be applied to the inside of the subject, to the one end of the light guide 4 under the control of the control apparatus 9. The light source apparatus 3 is implemented by at least one of light sources such as a Light Emitting Diode (LED) light source, a xenon lamp, and a semiconductor laser device including a Laser Diode (LD), a processor that is a processing apparatus that includes hardware, such as a Field Programmable Gate Array (FPGA) or a Central Processing Unit (CPU), and a memory that is a temporary storage area that is used by the processor. Meanwhile, the light source apparatus 3 and the control apparatus 9 may be configured to perform communication individually as illustrated in FIG. 1, or may be configured in an integrated manner.

The light guide 4 has the one end that is removably connected to the light source apparatus 3, and another end that is removably connected to the insertion portion 2. Further, the light guide 4 guides the illumination light that is supplied from the light source apparatus 3 from the one end to the other end and supplies the illumination light to the insertion portion 2.

An eyepiece portion 21 of the insertion portion 2 is removably connected to the camera head 5. Further, the camera head 5 receives the object image that is formed by the insertion portion 2, performs photoelectric conversion to form image data (RAW data), and outputs the image data to the control apparatus 9 via the first transmission cable 6, under the control of the control apparatus 9.

The insertion portion 2 and the camera head 5 described above correspond to an endoscope.

The first transmission cable 6 has one end that is removably connected to the control apparatus 9 via a video connector 61, and another end that is removably connected to the camera head 5 via a camera head connector 62. Further, the first transmission cable 6 transmits the image data that is output from the camera head 5 to the control apparatus 9, and transmits setting data, electric power, or the like that is output from the control apparatus 9 to the camera head 5. Here, the setting data is a control signal, a synchronous signal, a clock signal, or the like for controlling the camera head 5.

The display apparatus 7 is configured with a display monitor made of liquid crystal, organic Electro Luminescence (EL), or the like, and displays the display image based on the image data that is subjected to image processing by the control apparatus 9 and various kinds of information on the endoscope system 1 under the control of the control apparatus 9.

The second transmission cable 8 has one end that is removably connected to the display apparatus 7, and another end that is removably connected to the control apparatus 9. Further, the second transmission cable 8 transmits the image data that is subjected to image processing by the control apparatus 9 to the display apparatus 7.

The control apparatus 9 corresponds to a medical apparatus. The control apparatus 9 is implemented by a processor that is a processing apparatus that includes hardware, such as a Graphics Processing Unit (GPU), an FPGA, or a CPU, and a memory that is a temporary storage area that is used by the processor. Further, the control apparatus 9 comprehensively controls operation of the light source apparatus 3, the camera head 5, and the display apparatus 7 through the first transmission cable 6, the second transmission cable 8, and the third transmission cable 10 in accordance with a program that is recorded in the memory. Furthermore, the control apparatus 9 performs various kinds of image processing on the image data that is input via the first transmission cable 6 and outputs the image data to the second transmission cable 8.

The third transmission cable 10 has one end that is removably connected to the light source apparatus 3, and another end that is removably connected to the control apparatus 9. Further, the third transmission cable 10 transmits control data from the control apparatus 9 to the light source apparatus 3.

### Functional Configuration of Main Part of Endoscope System

A functional configuration of a main part of the endoscope system 1 as described above will be described below.

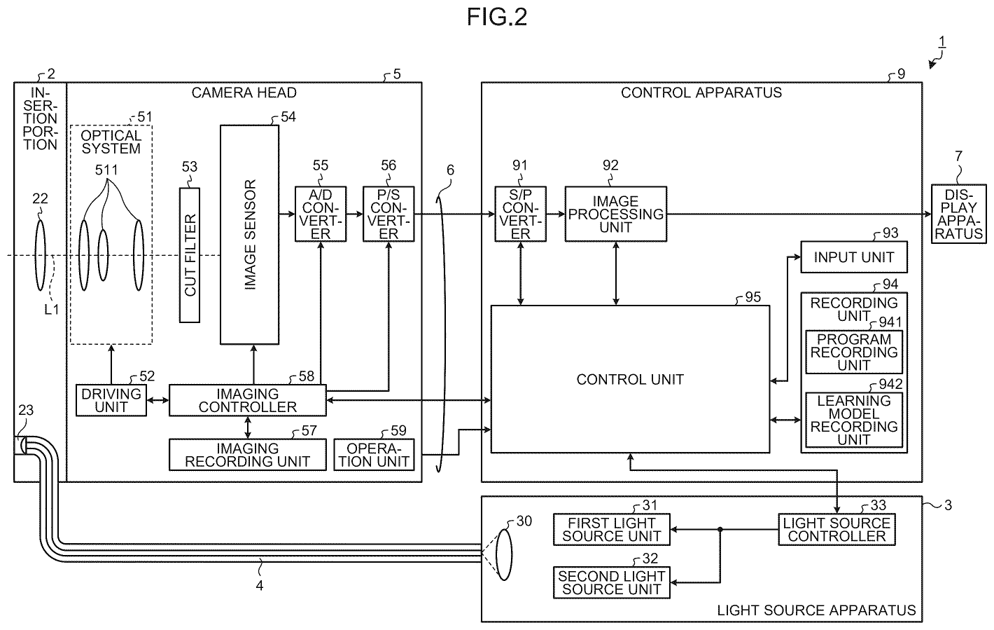

FIG. 2 is a block diagram illustrating a functional configuration of the main part of the endoscope system 1.

In the following, the insertion portion 2, the light source apparatus 3, the camera head 5, and the control apparatus 9 will be described in this order.

### Configuration of Insertion Portion

A configuration of the insertion portion 2 will be described below.

As illustrated in FIG. 2, the insertion portion 2 includes an optical system 22 and an illumination optical system 23.

The optical system 22 is configured with one or more lenses or the like, condenses reflected light that is reflected from an imaging object, return light that comes from the imaging object, excitation light that comes from the imaging object, fluorescence that is emitted by the imaging object, or the like, and forms an object image.

The illumination optical system 23 is configured with one or more lenses or the like, and applies illumination light that is supplied from the light guide 4 to the imaging object.

### Configuration of Light Source Apparatus

A configuration of the light source apparatus 3 will be described below.

As illustrated in FIG. 2, the light source apparatus 3 includes a condenser lens 30, a first light source unit 31, a second light source unit 32, and a light source controller 33.

The condenser lens 30 condenses light that is emitted by each of the first light source unit 31 and the second light source unit 32, and emits the condensed light to the light guide 4.

The first light source unit 31 emits white light (normal light) that is visible light and supplies the white light as illumination light to the light guide 4 under the control of the light source controller 33. The first light source unit 31 is configured with a collimator lens, a white LED lamp, a driving driver, and the like.

Meanwhile, the first light source unit 31 may supply white light that is visible light by causing a red LED lamp, a green LED lamp, and a blue LED lamp to simultaneously emit light. Further, the first light source unit 31 may of course be configured with a halogen lamp, a xenon lamp, or the like.

The second light source unit 32 emits excitation light in a predetermined wavelength band and supplies the excitation light as illumination light to the light guide 4 under the control of the light source controller 33.

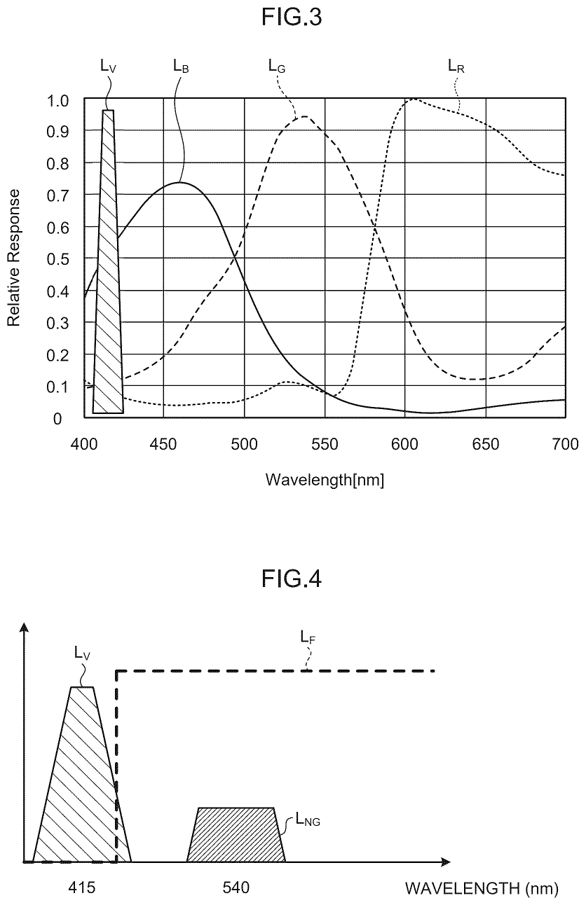

FIG. 3 is a diagram illustrating a wavelength characteristic of excitation light that is emitted by the second light source unit 32. Specifically, in FIG. 3, a horizontal axis represents a wavelength (nanometers (nm)) and a vertical axis represents the wavelength characteristic. Further, in FIG. 3, a curve L_{Y} represents a wavelength characteristic of the excitation light that is emitted by the second light source unit 32. Furthermore, in FIG. 3, a curve L_{B} represents a wavelength characteristic of a blue wavelength band, a curve L_{G} represents a wavelength characteristic of a green wavelength band, and a curve L_{R} represents a wavelength characteristic of a red wavelength band.

Here, as illustrated in FIG. 3, the second light source unit 32 emits excitation light in a wavelength band of 400 nm to 430 nm with a center wavelength (peak wavelength) of 415 nm. The second light source unit 32 is configured with a collimator lens, a semiconductor laser, such as a purple Laser Diode (LD), a driving driver, and the like.

The light source controller 33 is implemented by a processor that is a processing apparatus that includes hardware, such as an FPGA or a CPU, and a memory that is a temporary storage area that is used by the processor. Further, the light source controller 33 controls a light emission timing, a light emission duration, or the like of each of the first light source unit 31 and the second light source unit 32 based on control data that is input from the control apparatus 9.

### Configuration of Camera Head

A configuration of the camera head 5 will be described below.

As illustrated in FIG. 2, the camera head 5 includes an optical system 51, a driving unit 52, a cut filter 53, an image sensor 54, an analog-to-digital (A/D) converter 55, a parallel-to-serial (P/S) converter 56, an imaging recording unit 57, an imaging controller 58, and an operation unit 59.

The optical system 51 forms the object image that is condensed by the optical system 22 of the insertion portion 2 on a light receiving surface of the image sensor 54. The optical system 51 is configured with a plurality of lenses 511 (FIG. 2) such that a focal distance and a focal position are changeable. Specifically, the optical system 51 changes the focal distance and the focal position by causing the driving unit 52 to move each of the lenses 511 on an optical axis L1 (FIG. 2).

The driving unit 52 is configured with a motor, such as a stepping motor, a direct-current (DC) motor, or a voice coil motor, and a transmission mechanism, such as a gear, that transmits rotation of the motor to the optical system 51. Further, the driving unit 52 moves the plurality of lenses 511 of the optical system 51 along the optical axis L1 under the control of the imaging controller 58.

The cut filter 53 is arranged on the optical axis L1 between the optical system 51 and the image sensor 54. Further, the cut filter 53 blocks light in a predetermined wavelength band and transmits other light.

FIG. 4 is a diagram illustrating a transmission characteristic of the cut filter 53. Specifically, in FIG. 4, a horizontal axis represents a wavelength (nm) and a vertical axis represents a wavelength characteristic. Further, in FIG. 4, a curve LF represents a transmission characteristic of the cut filter 53, and a curve L_{V} represents a wavelength characteristic of excitation light. Furthermore, in FIG. 4, a curve LNG represents a wavelength characteristic of fluorescence that is generated by application of excitation light to Advanced Glycation End Products (AGEs) that are generated due to thermal treatment on a living tissue by an energy device or the like.

Here, as illustrated in FIG. 4, the cut filter 53 blocks a part of excitation light that is reflected from the living tissue in an observation area, and transmits light in other wavelength bands including a fluorescence component. More specifically, the cut filter 53 blocks a part of light in a wavelength band on a short wavelength side from 400 nm to less than 430 nm including excitation light, and transmits light in a wavelength band on a longer wavelength side than 430 nm including fluorescence that is generated by application of excitation light to AGEs that are generated due to thermal treatment.

The image sensor 54 is configured with an image sensor, such as a Charge Coupled Device (CCD) or a Complementary Metal Oxide Semiconductor (CMOS), in which any of color filters of Bayer arrangement (RGGB) is arranged on each of pixels that are arranged in a two-dimensional matrix manner. Further, the image sensor 54 receives an object image that is formed by the optical system 51 and that has passed through the cut filter 53, performs photoelectric conversion to generate image data (RAW data), and outputs the image data to the A/D converter 55, under the control of the imaging controller 58.

The A/D converter 55 is configured with an A/D conversion circuit or the like, performs A/D conversion processing on analog image data that is input from the image sensor 54, and outputs the image data to the P/S converter 56, under the control of the imaging controller 58.

The P/S converter 56 is configured with a P/S conversion circuit or the like, performs parallel-to-serial conversion on digital image data (corresponding to a captured image) that is input from the A/D converter 55, and outputs the image data to the control apparatus 9 via the first transmission cable 6, under the control of the imaging controller 58.

Meanwhile, it may be possible to arrange, instead of the P/S converter 56, an electrical-to-optical (E/O) converter that converts image data to an optical signal, and output the image data by the optical signal to the control apparatus 9. Further, for example, it may be possible to transmit the image data to the control apparatus 9 by radio communication, such as Wireless Fidelity (Wi-Fi) (registered trademark).

The imaging recording unit 57 is configured with a non-volatile memory or a volatile memory, and records therein various kinds of information on the camera head 5 (for example, pixel information on the image sensor 54 and characteristics of the cut filter 53). Further, the imaging recording unit 57 records therein various kinds of setting data and control parameters that are transmitted from the control apparatus 9 via the first transmission cable 6.

The imaging controller 58 is implemented by a Timing Generator (TG), a processor that is a processing apparatus that includes hardware, such as a CPU, and a memory that is a temporary storage area that is used by the processor. Further, the imaging controller 58 controls operation of each of the driving unit 52, the image sensor 54, the A/D converter 55, and the P/S converter 56 based on setting data that is received from the control apparatus 9 via the first transmission cable 6.

The operation unit 59 is configured with a button, a switch, or the like, receives user operation that is performed by a user, such as an operator, and outputs an operation signal corresponding to the user operation to the control apparatus 9. Examples of the user operation include operation of changing an observation mode of the endoscope system 1 to a normal light observation mode, a fluorescence observation mode, or a specific observation mode.

### Configuration of Control Apparatus

A configuration of the control apparatus 9 will be described below.

As illustrated in FIG. 2, the control apparatus 9 includes a serial-to-parallel (S/P) converter 91, an image processing unit 92, an input unit 93, a recording unit 94, and a control unit 95.

The S/P converter 91 performs serial-to-parallel conversion on the image data that is received from the camera head 5 via the first transmission cable 6 and outputs the image data to the image processing unit under the control of the control unit 95.

Meanwhile, when the camera head 5 outputs the image data by an optical signal, it may be possible to arrange, instead of the S/P converter 91, an optical-to-electrical (O/E) converter that converts an optical signal to an electrical signal. Further, when the camera head 5 transmits the image data by radio communication, it may be possible to arrange, instead of the S/P converter 91, a communication module that is able to receive a radio signal.

The image processing unit 92 is implemented by a processor that is a processing apparatus that includes hardware, such as a GPU or an FPGA, and a memory that is a temporary storage area that is used by the processor. Further, the image processing unit 92 performs predetermined image processing on image data of parallel data that is input from the S/P converter 91 and outputs the image data to the display apparatus 7 under the control of the control unit 95. Examples of the predetermined image processing include a demosaicing process, a white balance process, a gain adjustment process, a y correction process, a format conversion process, and a superimposition process.

The input unit 93 is configured with a mouse, a foot switch, a keyboard, a button, a switch, a touch panel, or the like, receives user operation that is performed by a user, such as an operator, and outputs an operation signal corresponding to the user operation to the control unit 95.

The recording unit 94 is configured with a volatile memory, a non-volatile memory, a Solid State Drive (SSD), a Hard Disk Drive (HDD), or a recording medium, such as a memory card. Further, the recording unit 94 records therein data that includes various kinds of parameters that are needed for operation of the endoscope system 1. Furthermore, the recording unit 94 includes a program recording unit 941 that records therein various kinds of programs for operating the endoscope system 1 and a learning model recording unit 942 as described below.

The learning model recording unit 942 records therein a learning model that is used for image recognition that is performed by the control unit 95. The learning model is a model that is generated by, for example, machine learning using Artificial Intelligence (AI).

Specifically, the learning model is a model that is obtained by adopting image data in which various kinds of areas, such as a stomach, an esophagus, and a duodenum, are captured as teacher data and performing machine learning (for example, deep learning or the like) on the area based on the teacher data.

The control unit 95 is implemented by a processor that is a processing apparatus that includes hardware, such as an FPGA or a CPU, and a memory that is a temporary storage area that is used by the processor. Further, the control unit 95 comprehensively controls each of the units included in the endoscope system 1.

The image processing unit 92 and the control unit 95 as described above correspond to a processor.

### Observation Principle in Observation Mode of Endoscope System

An observation principle in an observation mode of the endoscope system 1 will be described below.

In the following, a fluorescence observation mode and a normal light observation mode will be described in this order.

### Observation Principle in Fluorescence Observation Mode

An observation principle in the fluorescence observation mode will be described below.

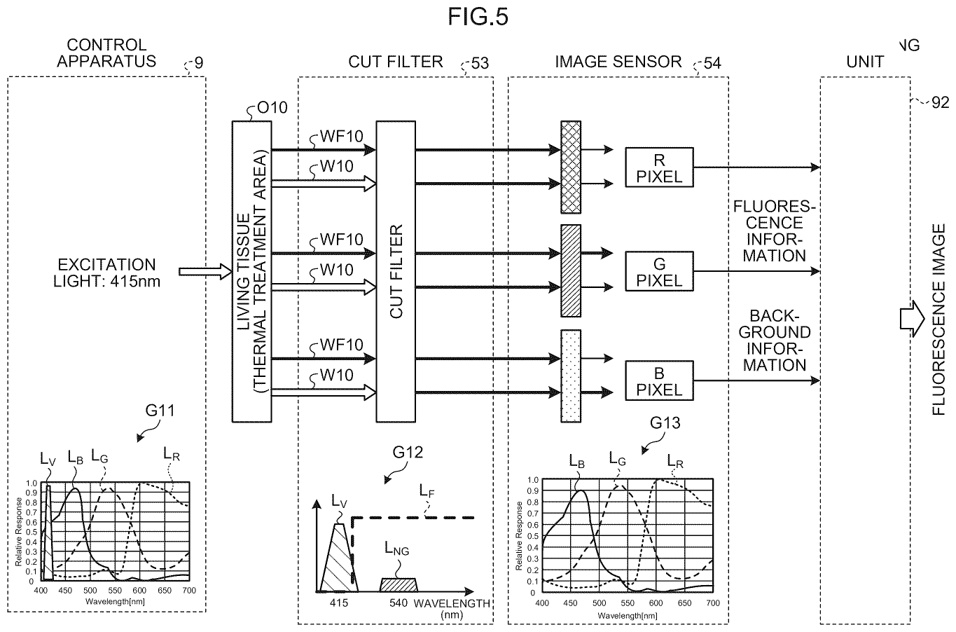

FIG. 5 is a diagram for explaining the observation principle in the fluorescence observation mode.

As illustrated in a graph G11 in FIG. 5, first, the light source apparatus 3 causes the second light source unit 32 to emit light to apply excitation light (center wavelength of 415 nm) to a living tissue O10 (thermal treatment area) in which thermal treatment is performed by an energy device or the like, under the control of the control apparatus 9. In this case, as illustrated in a graph G12 in FIG. 5, at least reflected light (hereinafter, described as reflected light W10), which includes a component of excitation light reflected by the living tissue O10 (thermal treatment area) and return light, is blocked by the cut filter 53 and intensity of the reflected light decreases, whereas a part of a component on a long-wavelength side as compared to the blocked wavelength band enters the image sensor 54 while retaining intensity.

More specifically, as illustrated in the graph G12 in FIG. 5, the cut filter 53 blocks a most part of the reflected light W10 that enters a G pixel of the image sensor 54, that is, the reflected light W10 in a wavelength band that includes the wavelength band of the excitation light and that is on the short-wavelength side, and transmits a wavelength band on the long-wavelength side as compared to the blocked wavelength band. Further, as illustrated in the graph G12 in FIG. 5, the cut filter 53 transmits fluorescence WF10 that AGEs in the living tissue O10 (thermal treatment area) have spontaneously emitted. Therefore, the reflected light W10 with reduced intensity and the fluorescence WF10 enter each of an R pixel, the G pixel, and a B pixel of the image sensor 54.

Here, the G pixel of the image sensor 54 has sensitivity against the fluorescence WF10. However, as represented by a curve LNG of a fluorescence characteristic in the graph G12 in FIG. 5, fluorescence reaction is minute. Therefore, an output value corresponding to the fluorescence WF10 in the G pixel is a small value.

Thereafter, the image processing unit 92 acquires image data (RAW data) from the image sensor 54, performs image processing on an output value of each of the G pixel and the B pixel that are included in the image data, and generates a fluorescence image. In this case, the output value of the G pixel includes fluorescence information corresponding to the fluorescence WF10 that is emitted from the thermal treatment area. Further, the output value of the B pixel includes background information from the living tissue of the subject including the thermal treatment area. Furthermore, by displaying the fluorescence image on the display apparatus 7, it is possible to observe the living tissue (thermal treatment area) that is subjected to thermal treatment by an energy device or the like.

### Observation Principle in Normal Light Observation Mode

An observation principle in the normal light observation mode will be described below.

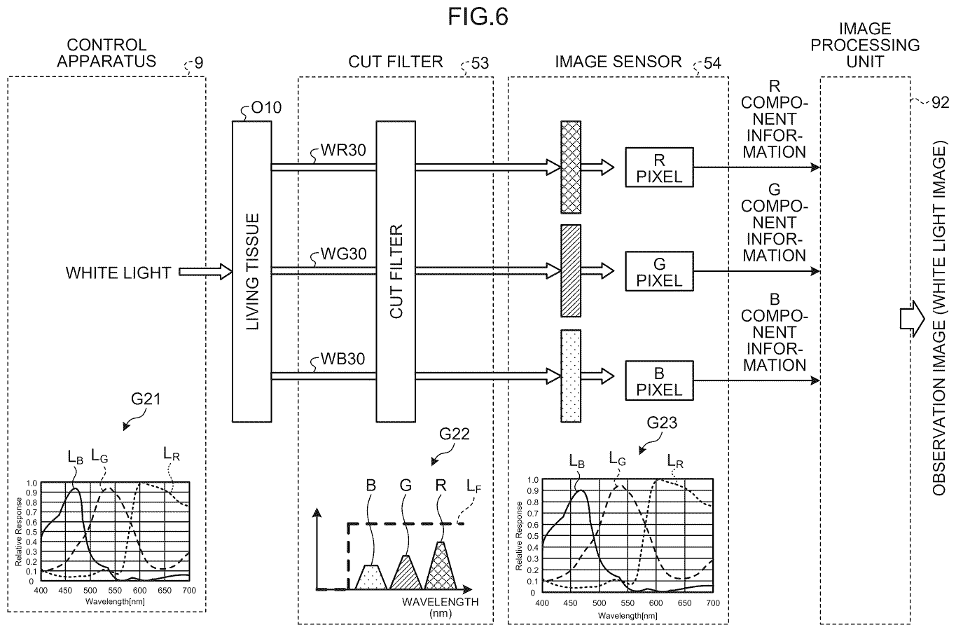

FIG. 6 a diagram for explaining the observation principle in the normal light observation mode.

As illustrated in a graph G21 in FIG. 6, first, the light source apparatus 3 causes the first light source unit 31 to emit light to apply white light to the living tissue O10 under the control of the control apparatus 9. In this case, a part of reflected light that is reflected by the living tissue O10 and return light (hereinafter, described as reflected light WR30, reflected light WG30, and reflected light WB30) is cut by the cut filter 53, and the rest of the light enters the image sensor 54. Specifically, as illustrated in a graph G22 in FIG. 6, the cut filter 53 blocks reflected light in a wavelength band that includes the wavelength band of the excitation light and that is on the short-wavelength side. Therefore, a component of light in the blue wavelength band that enters the B pixel of the image sensor 54 is reduced as compared to a state in which the cut filter 53 is not arranged.

Thereafter, the image processing unit 92 acquires image data (RAW data) from the image sensor 54, performs image processing on an output value of each of an R pixel, a G pixel, and a B pixel that are included in the image data, and generates an observation image (white light image). In this case, a blue component included in the image data is reduced as compared to the state in which the cut filter 53 is not arranged, and therefore, the image processing unit 92 performs a white balance adjustment process for adjusting a white balance so as to maintain a constant ratio among a red component, a green component, and a blue component. Further, by displaying the observation image (white light image) on the display apparatus 7, it is possible to observe a natural observation image (white light image) even when the cut filter 53 is arranged.

### Control Method

A control method that is performed by the control apparatus 9 will be described below.

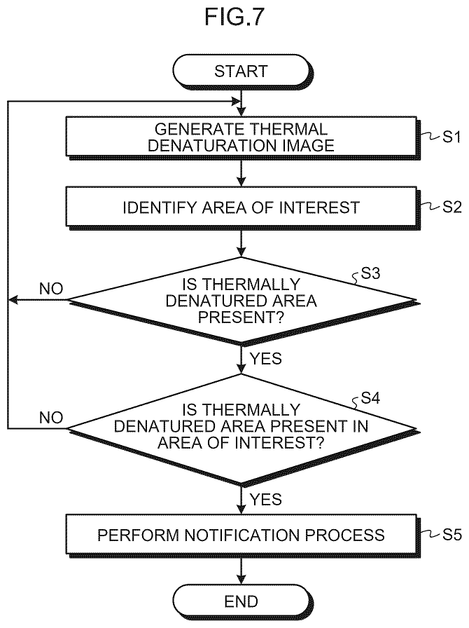

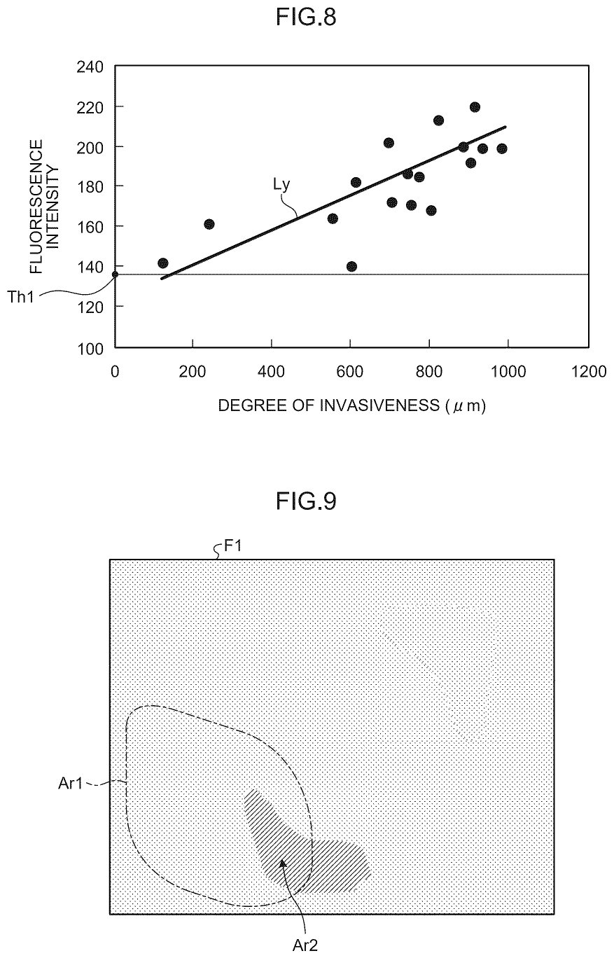

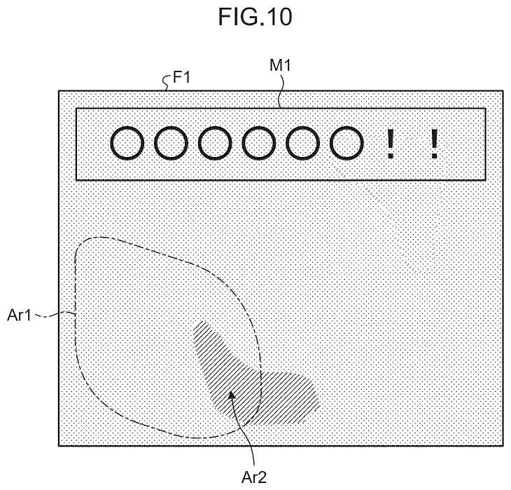

FIG. 7 is a flowchart illustrating a control method that is implemented by the control apparatus 9. FIG. 8 to FIG. 10 are diagrams for explaining the control method. Specifically, FIG. 8 is a diagram illustrating a correlation (a straight line L_{Y}) between fluorescence intensity of fluorescence that AGEs in the living tissue have spontaneously emitted and a degree of invasiveness (a depth and an area) due to the thermal treatment on the living tissue. Meanwhile, in FIG. 8, a vertical axis represents the fluorescence intensity and a horizontal axis represents the degree of invasiveness due to the thermal treatment on the living tissue. FIG. 9 is a diagram illustrating a thermal denaturation image F1 that is generated at Step S1. FIG. 10 is a diagram for explaining Step S5.

Meanwhile, in the following, it is assumed that the observation mode is changed to a specific observation mode by operation of “changing the observation mode of the endoscope system 1 to the specific observation mode” that is performed by a user, such as an operator, on the operation unit 59. Furthermore, it is assumed that area information that indicates an area of interest is input in advance by operation that is performed by the user, such as the operator, on the input unit 93.

Here, examples of the area information include information that directly indicates an area of an esophagus, a duodenum, or the like, and information indicating a procedure. For example, when association information for associating the procedure and the area is recorded in advance in the recording unit 94 or the like, the control unit 95 is able to refer to the association information and recognize an area that is designated by the user, such as the operator, from the information indicating the procedure (area information).

Firstly, the image processing unit 92 generates the thermal denaturation image F1 (FIG. 9) that makes it possible to identify a thermally denatured area Ar2 (FIG. 9) in which thermal denaturation has occurred in a living tissue, under the control of the control unit 95 (Step S1). Further, the thermal denaturation image F1 is displayed on the display apparatus 7. Here, in FIG. 9, the thermally denatured area Ar2 is represented as a shaded area.

Meanwhile, the fluorescence intensity of the fluorescence that the AGEs in the living tissue have spontaneously emitted and the degree of invasiveness (degree of thermal denaturation) due to a thermal process on the living tissue have a correlation with each other as illustrated in FIG. 8. Specifically, as represented by the straight line L_{Y} in FIG. 8, the fluorescence intensity increases with increase in the degree of thermal denaturation (with increase in the degree of invasiveness due to thermal treatment on the living tissue). Further, the thermally denatured area Ar2 is an area that is formed of pixels for each of which the fluorescence intensity of the fluorescence that AGEs in the living tissue have spontaneously emitted in the fluorescence image exceeds a specific fluorescence intensity Th1 (FIG. 8).

Specifically, in the specific observation mode, by alternately changing the fluorescence observation mode and the normal light observation mode, the fluorescence image and the observation image (white light image) are generated in a time-sharing manner. Further, the image processing unit 92 performs, at Step S1, a superimposition process of generating the thermal denaturation image F1 by superimposing the fluorescence image and the observation image (white light image) that is generated at an approximately same timing as the fluorescence image.

Here, examples of the superimposition process performed by the image processing unit 92 include a first superimposition process and a second superimposition process as described below.

The first superimposition process is a process of replacing, in the observation image (white light image), a certain area at the same pixel positions as the thermally denatured area Ar2 of the fluorescence image with an image of the thermally denatured area Ar2 of the fluorescence image.

The second superimposition process is a process (what is called an alpha blending process) of changing brightness of a color that represents fluorescence assigned to each of the pixels in the certain area of the observation image (white light image) at the same pixels positions as the thermally denatured area Ar2, in accordance with the fluorescence intensity at each of the pixel positions of the thermally denatured area Ar2 of the fluorescence image.

From the viewpoint as described above, the fluorescence image corresponds to a first captured image. Further, the observation image (white light image) corresponds to a second captured image.

After Step S1, the control unit 95 identifies an area of interest Ar1 (FIG. 9) in the thermal denaturation image F1 (Step S2).

Specifically, at Step S2, the control unit 95 recognizes an area that is designated by the user, such as the operator, based on area information that is input by operation that is performed by the user, such as the operator, on the input unit 93. Further, the control unit 95 identifies the area of interest Ar1 (for example, an esophagus, a duodenum, or the like) that corresponds to the recognized area based on the observation image (white light image) that is used to generate the thermal denaturation image F1, by image recognition using a learning model that is recorded in the learning model recording unit 942. Furthermore, the control unit 95 controls operation of the image processing unit 92, and, as illustrated in FIG. 9, generates an image in which the identified area of interest Ar1 is distinguishable from other areas in the thermal denaturation image F1. Moreover, the image in which the identified area of interest Ar1 is distinguishable from the other areas in the thermal denaturation image F1 is displayed on the display apparatus 7. Meanwhile, in FIG. 9, the area of interest Ar1 is represented by a dash-dotted line so as to be distinguishable from the other areas.

After Step S2, the control unit 95 determines whether or not the thermally denatured area Ar2 is present in the thermal denaturation image F1 (Step S3).

Specifically, at Step S3, the control unit 95 extracts, as the thermally denatured area Ar2, an area that is formed of pixels that exceed the specific fluorescence intensity Th1 from among all of pixels in the fluorescence image that is used to generate the thermal denaturation image F1. Here, examples of the fluorescence intensity that is used at Step S3 include at least a g value in a pixel value of (r, g, b) of each of the pixels of the fluorescence image that is subjected to a demosaicing process and a luminance value that corresponds to a Y signal (luminance signal).

Meanwhile, at Step S3, the thermally denatured area Ar2 is extracted based on the fluorescence image, but embodiments are not limited to this example. For example, it may be possible to extract the thermally denatured area Ar2 based on image data that is not yet subjected to image processing by the image processing unit 92. In this case, examples of the fluorescence intensity include an output value of the G pixel in the image sensor 54.

Furthermore, when the thermally denatured area Ar2 is extracted, the control unit 95 determines as “Yes” at Step S3. In contrast, when the thermally denatured area Ar2 is not extracted, the control unit 95 determines as “No” at Step S3.

When it is determined that the thermally denatured area Ar2 is not present in the thermal denaturation image F1 (Step S3: No), the control apparatus 9 returns to Step 1.

In contrast, when determining that the thermally denatured area Ar2 is present in the thermal denaturation image F1 (Step S3: Yes), the control unit 95 determines whether or not the thermally denatured area Ar2 is present in the area of interest Ar1 (Step S4).

When determining that the thermally denatured area Ar2 is not present in the area of interest Ar1 (Step S4: No), the control apparatus 9 returns to Step 1.

In contrast, when determining that the thermally denatured area Ar2 is present in the area of interest Ar1 (Step S4: Yes), the control unit 95 performs a notification process (Step S5).

Specifically, at Step S5, the control unit 95 controls operation of the image processing unit 92, and, as illustrated in FIG. 10, generates an image in which warning information M1 indicating that the thermally denatured area Ar2 is present in the area of interest Ar1 is superimposed at a predetermined position in the thermal denaturation image F1. Further, the image in which the warning information M1 is superimposed at the predetermined position in the thermal denaturation image F1 is displayed on the display apparatus 7. In other words, the display apparatus 7 corresponds to a notification unit.

According to one embodiment as described above, it is possible to achieve effects as described below.

In the control apparatus 9 according to one embodiment, the control unit 95 identifies the area of interest Ar1 in the thermal denaturation image F1, and extracts the thermally denatured area Ar2 in the thermal denaturation image F1. Further, the control unit 95 determines whether or not the thermally denatured area Ar2 is present in the area of interest Ar1, and when determining that the thermally denatured area Ar2 is present in the area of interest Ar1, causes the display apparatus 7 to provide the specific information M1.

Therefore, for example, when surgical operation for stomach cancer is performed, it is possible to allow the user, such as the operator, to recognize whether or not an adjacent organ, such as an esophagus or a duodenum, that is adjacent to a stomach that is an operation target is affected by thermal invasion at the time of treatment by an energy device or the like.

Consequently, according to the control apparatus 9 of one embodiment, it is possible to allow the user, such as the operator, to recognize whether or not an unintended area in a living tissue is affected by thermal invasion, so that it is possible to improve convenience.

Furthermore, in the control apparatus 9 according to one embodiment, the control unit 95 extracts, as the thermally denatured area Ar2, an area that is formed of pixels that exceed the specific fluorescence intensity Th1 among all of pixels of the fluorescence image that is used to generate the thermal denaturation image F1.

Therefore, it is possible to easily extract the thermally denatured area Ar2 with high accuracy.

Moreover, in the control apparatus 9 according to one embodiment, the control unit 95 identifies the area of interest Ar1 based on the observation image (white light image) that is used to generate the thermal denaturation image F1.

Specifically, the area of interest Ar1 is identified based on the observation image (white light image) in which a feature of the living tissue in the observation area is clarified, so that it is possible to identify the area of interest Ar1 with high accuracy.

### OTHER EMBODIMENTS

While embodiments for carrying out the disclosure have been described above, the disclosure is not limited by the embodiments as described above.

In one embodiment as described above, the medical apparatus is mounted on an endoscope system using a rigid endoscope, but embodiments are not limited to this example, and may be mounted on an endoscope system using a flexible endoscope or an endoscope system using a medical surgical robot.

In one embodiment as described above, the control unit 95 may have a function as a learning unit of a learning apparatus. In this case, the control apparatus 9 corresponds to a learning apparatus.

Specifically, the control unit 95 generates a trained model by performing machine learning by using teacher data in which a fluorescence image that is obtained by applying excitation light to a living tissue and imaging fluorescence that is emitted from the living tissue and a white light image that is obtained by applying white light to the living tissue and imaging the living tissue are adopted as input data and information on determination whether or not a thermally denatured area is present in an area of interest is adopted as output data.

Here, the trained model is formed of a neural network in which each of layers includes one or a plurality of nodes. Further, a type of the machine learning is not specifically limited; however, for example, it is sufficient to prepare teacher data and training data in which fluorescence images and white light images of a plurality of subjects are associated with information on determination on whether or not a thermally denatured area is present in an area of interest in the plurality of fluorescence images and white light images, and perform learning by inputting the teacher data and the training data in a calculation model that is based on a multi-layer neural network. Furthermore, as a method of the machine learning, for example, a method based on a Deep Neural Network (DNN) as a multi-layer neural network, such as a Convolutional Neural Network (CNN) or a 3D-CNN, may be used. Moreover, as the method of the machine learning, a method based on a Recurrent Neural Network (RNN), a Long Short-Term Memory units (LSTM) in which the RNN is expanded, or the like may be used. Meanwhile, a learning unit of a different learning apparatus from the control apparatus 9 may implement the above-described functions.

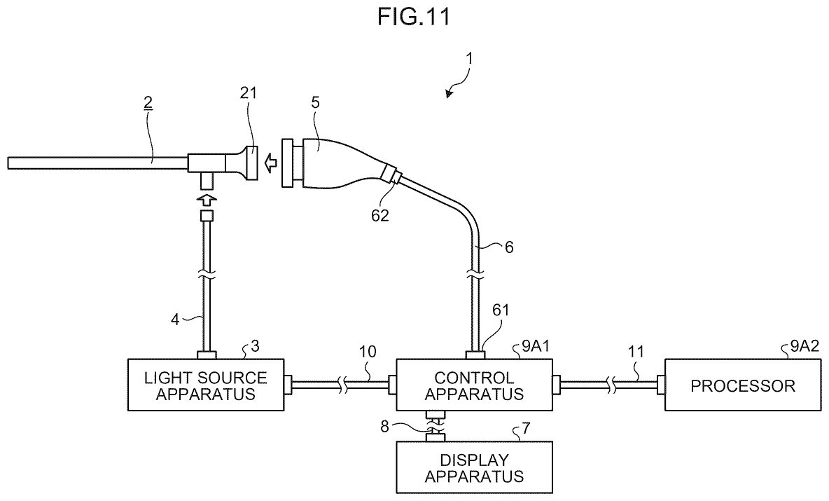

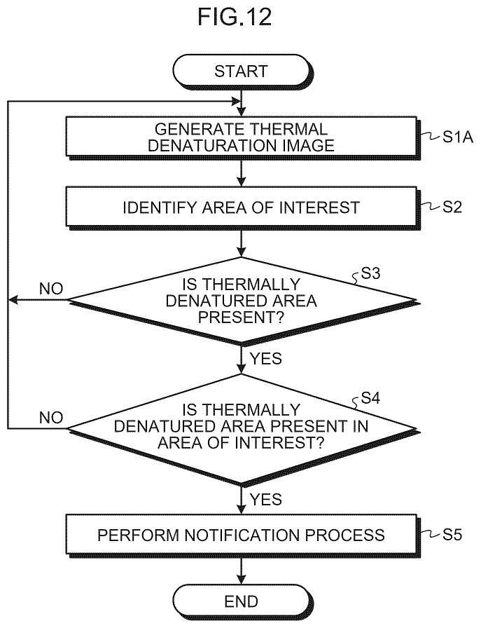

FIG. 11 and FIG. 12 are diagrams for explaining a modification of one embodiment. Specifically, FIG. 11 is a diagram corresponding to FIG. 1. FIG. 12 is a diagram corresponding to FIG. 7.

In one embodiment as described above, the control apparatus 9 that corresponds to the medical apparatus generates the thermal denaturation image F1, but embodiments are not limited to this example, and it may be possible to adopt the configuration of the endoscope system 1 according to one modification.

The endoscope system 1 according to one modification includes a processor 9A2 and a fourth transmission cable 11. Meanwhile, for distinction from the control apparatus 9 of one embodiment as described above, the control apparatus 9 according to one modification is described as a control apparatus 9A1.

In the control apparatus 9A1, functions for performing the processes from Step S2 to Step S5 among the functions of the control unit 95 are omitted as compared to the control apparatus 9 of one embodiment as described above.

The processor 9A2 corresponds to the medical apparatus and the processor. The processor 9A2 is implemented by a processor that is a processing apparatus that includes hardware, such as a GPU, an FPGA, or a CPU, and a memory that is a temporary storage area that is used by the processor. Further, the processor 9A2 is connected so as to be able to input and output information with respect to the control apparatus 9A1 via the fourth transmission cable 11. Furthermore, the processor 9A2 has functions to perform the processes from Step S2 to Step S5 among the functions of the control unit 95 of one embodiment as described above. Moreover, the processor 9A2 records therein information that is needed to perform the processes from Step S2 to Step S5. Furthermore, the processor 9A2 acquires, from the control apparatus 9A1, the thermal denaturation image F1 that is generated by the control apparatus 9A1 (Step S1A), and performs the processes from Step S2 to Step S5.

In one embodiment as described above, the control unit 95 identifies the area of interest Ar1 by image recognition using the learning model, but embodiments are not limited to this example.

For example, the control unit 95 generates a three-dimensional image (hereinafter, described as a Computer Graphics (CG) image) by using a well-known method (for example, volume rendering or the like) from a plurality of tomographic images that are captured by a tomography apparatus, such as a Computed Tomography (CT) or Magnetic Resonance Imaging (MRI) (for example, see Japanese Patent No. 6990292). Furthermore, the user, such as the operator, inputs the area information that indicates an area of interest (three-dimensional coordinates indicating the area of interest) by using the input unit 93 while checking the CG image that is displayed on the display apparatus 7. Moreover, the control unit 95 identifies, as the area of interest Ar1, an area that corresponds to a certain shape that represents the area of interest based on the area information, from a feature amount that indicates the shape of the observation image (white light image) based on the observation image (white light image) that is used to generate the thermal denaturation image F1.

Meanwhile, when identifying the area of interest Ar1, it is not needed to always adopt a configuration in which the feature amount that indicates the shape of the observation image (white light image) is used as described above, but it may be possible to adopt a different configuration.

For example, the camera head 5 may be configured with a stereo camera. Further, a stereo measurement technology for causing the stereo camera to simultaneously capture images from different viewpoints and calculating three-dimensional coordinates of an imaging object based on the principle of triangulation by using an amount of relative deviation between the images of the same imaging object. Furthermore, the control unit 95 identifies, as the area of interest Ar1, an area at three-dimensional coordinates corresponding to the three-dimensional coordinates indicating the area of interest in the area information, in the observation image (white light image) which is captured by the stereo camera and for which the three-dimensional coordinates are identified.

Moreover, for example, the camera head 5 is configured with a distance image sensor, such as a Time Of Flight (TOF) sensor or the like. Furthermore, the control unit 95 identifies, as the area of interest Ar1, an area at three-dimensional coordinates corresponding to the three-dimensional coordinates that represent the area of interest in the area information, in the observation image (white light image) which is captured by the distance image sensor and for which the three-dimensional coordinates are identified.

In one embodiment as described above, the display apparatus 7 is adopted as the notification unit, but embodiments are not limited to this example. As the notification unit, it may be possible to adopt a different configuration, such as a speaker, that gives a notice by voice, instead of the configuration that gives a notice by displaying an image. Furthermore, in one embodiment as described above, it may be possible to display the thermally denatured area Ar2 that is present in the area of interest Ar1 and the thermally denatured area Ar2 that is present outside the area of interest Ar1 in different modes on the display apparatus 7.

In one embodiment as described above, the area that is formed of the pixels that exceed the specific fluorescence intensity Th1 among pixels of the fluorescence image that is used to generate the thermal denaturation image F1 is extracted as the thermally denatured area Ar2, but embodiments are not limited to this example. For example, it may be possible to extract, as the thermally denatured area Ar2, an area that is formed of pixels for each of which a difference in the fluorescence intensity between corresponding pixels in two fluorescence images one of which is temporally prior to the other is equal to or larger than a specific threshold.

In one embodiment as described above, the image in which the observation image (white light image) and fluorescence image that are generated at approximately the same timing are superimposed is adopted as the thermal denaturation image, but embodiments are not limited to this example. For example, it may be possible to adopt, as the thermal denaturation image, the fluorescence image itself, instead of the superimposed image as described above.

According to one aspect of a medical apparatus, an endoscope system, a control method, a control program, and a learning apparatus, it is possible to improve convenience.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A medical apparatus comprising:
a processor comprising hardware, the processor being configured to
acquire area information that includes information on an area of interest based on input from a user, the area of interest being an organ that is adjacent to a target organ that is a target for thermal treatment,
acquire a fluorescence image that is generated based on an imaging signal captured at a timing of application of excitation light,
extract a thermally denatured area based on the fluorescence image, and
generate thermal denaturation information based on the area of interest and the thermally denatured area.

2. The medical apparatus according to claim 1, wherein the processor is further configured to extract the thermally denatured area based on a fluorescence intensity of each of pixels of the fluorescence image.

3. The medical apparatus according to claim 1, wherein the processor is further configured to extract, as the thermally denatured area, an area that is formed of pixels that exceed a specific fluorescence intensity among all of pixels of the fluorescence image.

4. The medical apparatus according to claim 1, wherein the processor is further configured to extract the thermally denatured area based on a difference in fluorescence intensity between corresponding pixels in two fluorescence images one of which is temporally prior to the other.

5. The medical apparatus according to claim 1, wherein the thermal denaturation information includes information that indicates the thermally denatured area that is present in the area of interest.

6. The medical apparatus according to claim 1, wherein the thermal denaturation information includes information that indicates the thermally denatured area that is present outside the area of interest.

7. The medical apparatus according to claim 1, wherein the thermal denaturation information includes information that indicates the thermally denatured area that is present in the target organ.

8. The medical apparatus according to claim 1, wherein the target organ is a stomach.

9. The medical apparatus according to claim 1, wherein the area of interest is an area corresponding to an esophagus, a duodenum, or the esophagus and the duodenum.

10. The medical apparatus according to claim 1, wherein the processor is further configured to generate a white light image based on an imaging signal that is captured at a timing of application of white light.

11. The medical apparatus according to claim 10, wherein the thermal denaturation information is an image in which information indicating the thermally denatured area that is present in the area of interest is superimposed on the white light image.

12. The medical apparatus according to claim 1, wherein the thermal denaturation information is an image in which a thermally denatured area that is present in the area of interest and a thermally denatured area that is present outside the area of interest are displayed in a distinguishable manner on a white light image.

13. The medical apparatus according to claim 1, wherein
the input from the user is information on a procedure, and
the processor is further configured to acquire the area information based on the information on the procedure.

14. The medical apparatus according to claim 13, further comprising:
a memory configured to store therein association information in which the procedure and the area information are associated with each other, and
the processor is further configured to acquire the area information based on the information on the procedure that is input from the user and the association information.

15. A medical system comprising:
an endoscope that includes an image sensor;
a light source apparatus that includes a light source configured to apply white light and excitation light; and
a control apparatus that includes a processor comprising hardware, the processor being configured to
acquire area information that includes information on an area of interest based on input from a user, the area of interest being an organ that is adjacent to a target organ that is a target for thermal treatment,
acquire a fluorescence image that is generated based on an imaging signal captured at a timing of application of excitation light,
extracts a thermally denatured area based on the fluorescence image, and
generate thermal denaturation information based on the area of interest and the thermally denatured area.

16. A control method of controlling a medical apparatus, the control method comprising:
acquiring area information that includes information on an area of interest based on input from a user, the area of interest being an organ that is adjacent to a target organ that is a target for thermal treatment;
acquiring a fluorescence image that is generated based on an imaging signal captured at a timing of application of excitation light;
extracting a thermally denatured area based on the fluorescence image; and
generating thermal denaturation information based on the area of interest and the thermally denatured area.

17. A non-transitory computer-readable recording medium with an executable program stored thereon, the program causing a processor of a medical device to execute:
acquiring area information that includes information on an area of interest based on input from a user, the area of interest being an organ that is adjacent to a target organ that is a target for thermal treatment;
acquiring a fluorescence image that is generated based on an imaging signal that is captured at a timing of application of excitation light;
extracting a thermally denatured area based on the fluorescence image; and
generating thermal denaturation information based on the area of interest and the thermally denatured area.
